(19) United States
(12) Patent Application Publication
(10) Pub. No.: US 2011/0253131 A1
(43) Pub. Date: Oct. 20, 2011

(54) Apparatus for the administration of pharmaceutical products in aerosol form

(76) Inventor: Paolo Licciulli, Legano (IT)

(21) Appl. No.: 13/068,379

(22) Filed: May 10, 2011

Related U.S. Application Data
(63) Continuation of application No. 11/886,133, filed on Sep. 10, 2007.

(30) Foreign Application Priority Data
Mar. 24, 2005 (IT) MI2005A000491

(51) Int. Cl. A61M 11/00

(52) U.S. Cl. 128/200.14

(57) ABSTRACT

An apparatus (10) is described for the administration of pharmaceutical products in the form of an aerosol, comprising means for generating a constant flow (11) of air; means for generating a pre-established flow of air (14); means for conveying the air flow (15) to the aerial tracts of a patient, means for containing the pharmaceutical product to be nebulized (12), wherein said apparatus (10) envisages means for generating a pre-established flow of air during all expiration phases of the respiratory cycle, said means for generating a pre-established air flow being pneumatically connected with the means for conveying the air flow (15).

The present invention relates to an apparatus for the administration of pharmaceutical products in aerosol form or nebulized.

As is known, the respiratory system, comprising lungs and chest, operates as a pump which rests on a muscle called diaphragm, which coordinates the inspiration and expiration actions of air and oxygen. In the natural respiratory process, the contraction of the diaphragm generates a vacuum in the lungs, which causes the inspiration of air from the outside.

As is known, there are numerous affections of the respiratory system which diminish the respiratory capacity of the patient.

In the case, for example, of patients suffering from asthma or one of the many other lung affections, which require the administration of suitable curative products in the bronchi and lung alveoli, it should be remembered that, in effecting said administration, there is a clear decrease in the respiratory capacity of the patient. There are currently three main methods for the administration of aerosol or drug treatment to these patients, i.e. (1) by means of atomizers of the Venturi-jet type or of the ultrasonic piezoelectric type, which produce aerosols from drug solutions; (2) by means of inhalators of measured dosages (MDI) consisting of pressurized cylinders with fluorocarbides or other gases and (3) dry powder inhalators.

Devices of type (1) or moist atomizers are jet nebulizers which exploit the Venturi principle: the energy source is compressed air which also serves to direct the aerosol towards the spontaneous respiration of the patient. In jet nebulizers, the oxygen and/or air flow creates the aerosol, through the device, starting from the drug in solution and carries it towards the patient, who can then breathe it from a mask or mouthpiece.

The use of the respiratory tract for the administration of drugs is becoming increasingly more important. In this type of approach, not only has the application of drug products which produce local effects in the treatment of lung affections been developed, but new strategies have also been envisaged, which use the lungs as the organ through which the drugs enter the body and produce systemic effects.

The use of this system, inhalation, for the administration of drugs has led to an ever-growing demand for an improvement in the quality of inhalation, which is not sufficient with the devices currently existing on the market.

At present, in fact, devices of the jet nebulizer type are absolutely inefficient as only 20% of the product, in the form of an aerosol, reaches the inner recesses of the lungs.

This is due to the fact that the efficiency of the existing devices depends on the conditions of the patient and on his respiratory capacity. It is the patient, in fact, that carries the product, nebulized by a pump starting from a liquid in a Venturi basin, towards the aerial tract, by means of his respiratory capacity. It is evident that a patient with a reduced respiratory capacity cannot effect a sufficient transmission of the nebulized drug.

As mentioned above, the penetration of the nebulized product is limited and 80% of the drug remains in the first respiratory tract.

It is evident that, if the pathology to be cured is a pathology of the first respiratory tract, the efficiency is good, otherwise the aerosol treatment is practically null.

Furthermore, it should be remembered that the rest of the vapour passes from the throat to the stomach, with relevant side-effects in the case of the use of particular active principles.

An increase in the dosage in order to compensate the inefficiency of the nebulizer can be harmful for the patient's health.

A general objective of the present invention is therefore to solve the above-mentioned drawbacks.

An object of the present invention is an apparatus for the administration of pharmaceutical products in the form of an aerosol, comprising means for generating a constant air flow; means for generating a pre-determined air flow; means for conveying the air flow to the aerial tracts of a patient; means for containing the pharmaceutical product to be nebulized, said equipment being characterized in that it includes means for generating a pre-established air flow during all expiration phases of the respiratory cycle, said means for generating a pre-established air flow being in pneumatic connection with the means for conveying the air flow.

The means for generating a constant air flow preferably consist of a pump and the means for generating a pre-established air flow during all expiration phases also preferably consist of a resistance pump which synchronizes the administration of a pre-established flow of air with the expiration phase.

The apparatus according to the present invention can also be used without a pharmaceutical product.

The means for conveying the air flow to the aerial tracts of a patient comprise a connector and a mask or mouthpiece, said means for generating a pre-established flow of air being operatively connected to said connector or to said mask or mouthpiece, by the means for conveying the air flow.

In particular, the first pump has the objective of nebulizing the drug as a common aerosol, the second, on the contrary, has the objective of supplying a flow of air which, at the right moment, intervenes on the normal ventilation flow by varying its rate and resistances with the purpose of obtaining a greater and better penetration of the drug towards the small aerial tract and stimulating a movement of the large aerial tracts so as to favour expectoration.

The main advantage of the apparatus according to the invention, in fact, consists not only in allowing therapies similar to those of the aerosol apparatuses already on the market to be effected, i.e. inhalation therapies effective for the first aerial tract, but also providing inhalation therapies effective towards the small aerial tracts.

Thanks to the improved efficiency of the apparatus, only partially dependent on the patient's respiratory capacity, it is also evident that lower dosages of the drug can be used, which will be better distributed.

A further advantage of the apparatus according to the present invention, moreover, is that it also effects a lung exercise, which is already effective even without drugs.

By exploiting the elastic capacities of the aerial tracts, by the use of a resistant pressure in the expiration phase, the apparatus according to the present invention compels the aerial tracts to slightly dilate until the end of the expiration. At the end of this phase, the resistant pressure is interrupted, thus relaxing the walls of the respiratory tracts: (the resistant pressure is interrupted at any point of the expiration phase, according to necessity).

This movement facilitates the inlet of air in the subsequent inspiration phase. The sequence of these movements allows a better change of air, the detachment of mucus and secretions, as well as the penetration of the drug.

It is also a “natural” movement, which follows the physiological characteristics of the patient's respiration, as the functioning of the apparatus is regulated by the manner and frequency of the patient's respiration.

A further embodiment of the apparatus according to the present invention envisages that the means for generating the pre-established air flow consist of a first resistant pump which synchronizes the administration of a pre-established flow of air with the expiration phase and a second resistant pump which works continuously, administering a pre-established flow of air during the two inspiration and expiration respiratory phases at a constant pressure. The resistant pump operates in continuous at a pressure of 1 cm of water.

The presence of this second resistant pump allows a better expectoration of the patient.

The structural and functional characteristics of the present invention and its advantages with respect to the known art will appear even more evident by examining the following description referring to the enclosed drawings, in which:

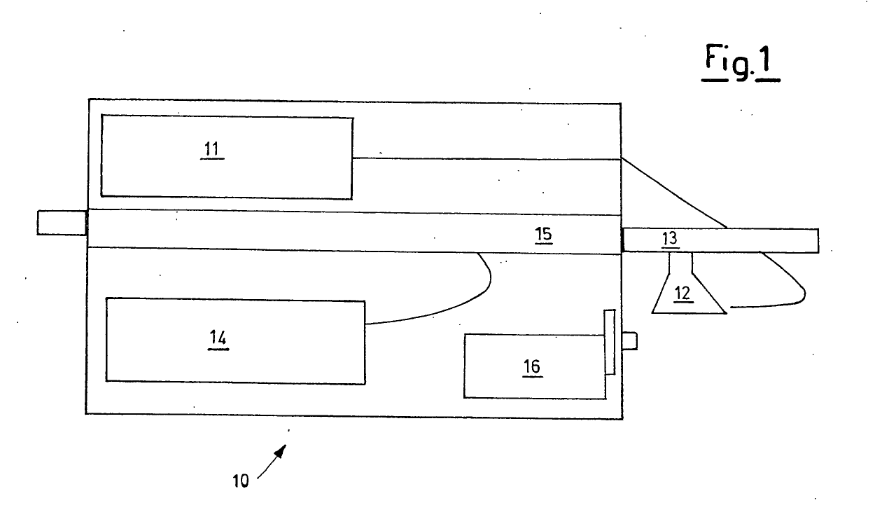

FIG. 1 is a schematic representation of an embodiment of the apparatus according the present invention;

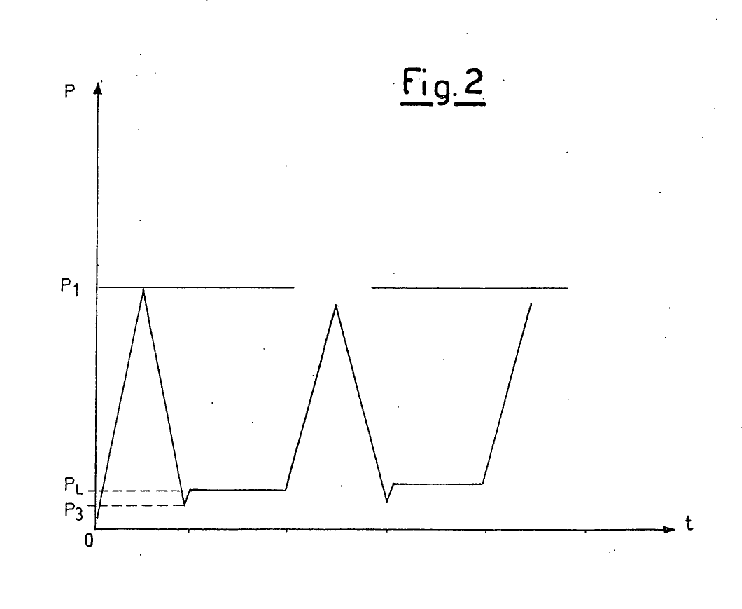

FIG. 2 is a schematic representation of the pressure variation with the variation in time in an apparatus such as that represented in FIG. 1;

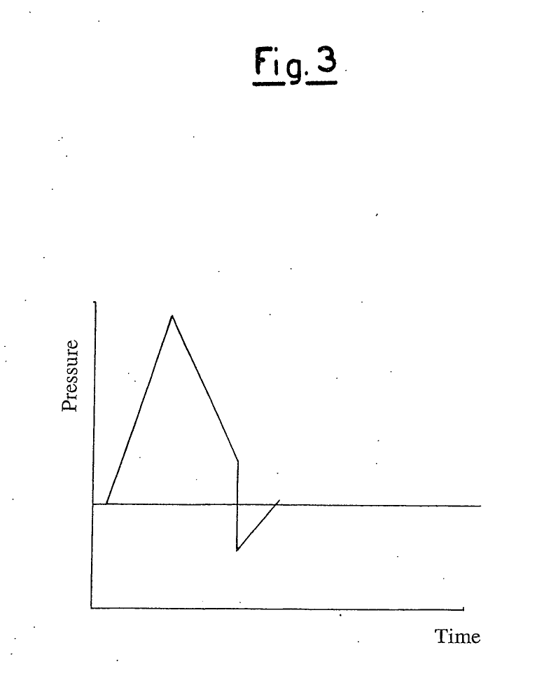

FIG. 3 is a schematic representation of the pressure variation in relation to time in a respiration cycle of a patient subjected to treatment with the apparatus according to the present invention, which envisages the first resistant pump only;

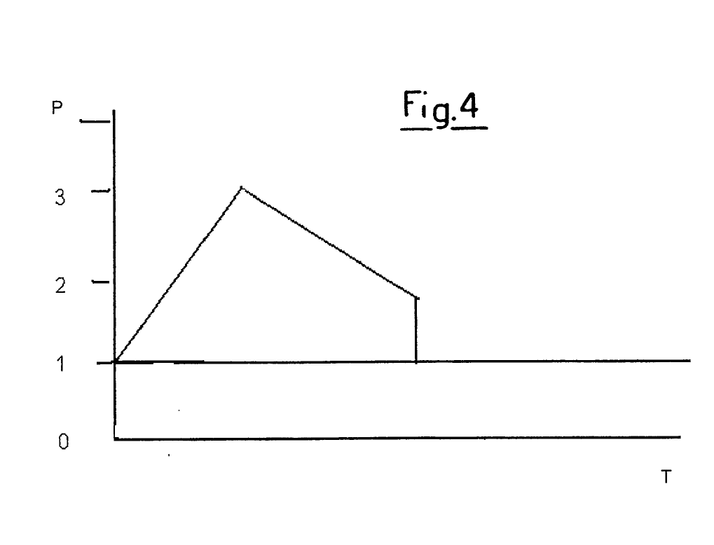

FIG. 4 is a schematic representation of the pressure variation in relation to time in a respiration cycle of a patient subjected to treatment with the apparatus according to the embodiment of the present invention, which envisages also the second resistant pump operating in continuous.

In particular, FIG. 1 shows an embodiment of the apparatus according to the present invention, wherein 10 indicates the apparatus. It includes a nebulization pump 11 which generates a constant air flow, which effects the nebulization of the liquid solution of the pharmaceutical product contained in 12, i.e. in a container consisting of a Venturi basin. More specifically, the constant air flow generated by the pump 11, is directly sent to the nebulization basin 12.

This basin is then connected, through a tubular connection 13, to a mask or mouthpiece (not shown in the figure), worn by the patient and through which the nebulized product enters the aerial tract of the patient.

The apparatus 10, in the embodiment shown in FIG. 1, also envisages a second pump indicated as 14, connected, through suitable means 15 for conveying the air flow, to the aerial tracts of a patient. This second pump 14 generates a resistant pressure, supplied by means of a flow of compressed air, contrary in the expiration phase. The means 15 for conveying the flow of air to the aerial tracts of the patient is connected, again through the tubular connection 13, to the mask or mouthpiece (not shown in the figure) worn by the patient and through which the nebulized product enters the aerial tracts of the patient.

The apparatus shown in FIG. 1 also includes a handling and control system of the pumps 16, which, in the embodiment represented in the figure, is a potentiometer.

The air flow generated by the pumps used in the apparatus according to the present invention is preferably a flow of 10 liters per minute.

In particular, the means for conveying the air flow to the patient 15 is pneumatically connected with the fitting 13 and then with the mask, so as to administrate a certain quantity of air in the expiration flow. The air expired by the patient passes through the fitting 13 to the duct 15.

The patient consequently breathes naturally in all phases of the respiratory cycle, receiving a pressure of positive air supplied by the apparatus due to the action of the pump 11 with the nebulized pharmaceutical product, during the inspiration phase and a pressure of positive air during the expiration phase due to the combined action of the pump 11 and the resistant pump 14.

The apparatus according to the present invention, schematically shown in FIG. 1, shows a pressure variation in relation to the time in a respiratory cycle with alternating inspiration and expiration phases, said variation being schematically shown in FIG. 2.

In particular, in the apparatus 1 according to the present invention, P.sub.1 represents the pressure at the beginning of the expiration phase, whereas the final pressure of the expiration phase P.sub.3 is lower than the pressure P.sub.2 of the beginning of the subsequent inspiration phase.

The slight depression caused by the pressure drop between P.sub.2 and P.sub.3 is sufficient for creating a vortex in the aerial tracts of the patient, which allows the air flow of the subsequent inspiration phase to also reach the most peripheral aerial tracts, i.e. the small aerial tracts. This pressure drop is generated by the switching off of the resistant pump 14.

In this way the apparatus 1 according to the present invention, by exploiting the elastic properties of the aerial tracts, with the use of a resistant pressure during the expiration phase, forces a slight dilatation upon the aerial tracts, which reaches the end of the expiration. At the end of this phase, the resistant pressure is interrupted, thus relaxing the respiratory tracts.

This movement facilitates the inlet of air during the subsequent inspiration phase. The succession of these movements allows a better change of air, the detachment of mucus and secretions and penetration of the drug. A better penetration of the pharmaceutical product towards the small aerial tracts is in fact obtained together with a stimulation of movement in the large aerial tracts, thus favouring expectoration.

Thanks to this movement, the apparatus according to the present invention also effects a lung exercise which is already efficacious even without drugs.

FIG. 3 shows the pressure variation in relation to the time in a respiration cycle of a patient subjected to treatment with the apparatus according to the present invention, which includes a single resistant pump. In particular, it shows the pressure variation per volume of air (from 0 to 10 cm of water), wherein the respiration pressure and the counter-pressure drop are evident.

FIG. 4 shows the pressure variation in relation to the time in a respiration cycle of a patient subjected to treatment with the apparatus according to the embodiment of the present invention which includes a second resistant pump. In particular, the pressure variation is shown per volume of air (from 1 to 10 cm of water), wherein the respiration pressure and the counter-pressure drop are evident.

1-7. (canceled)

8. A method of administering a pharmaceutical product in the form of an aerosol, said method comprising:
(a) generating a constant flow of air by a the use of a nebulization pump that is operative during inspiration and expiration phases of a respiratory cycle;
(b) generating a pre-established flow of air by a resistant pump wherein said nebuluization pump and said resistant pump are connected to a common duct an said common duct adapted for conveying an air flow to aerial tracts of a patient;
(c) providing a nebulization basin containing a pharmaceutical product to be nebulized by said pre-established flow of air in said common duct;
(d) reducing said pre-established air flow at said resistant pump to provide a flow of air during all expiration phases of a respiratory cycle where said air flow is lower during the expiration phase than during the inspiration phase.

9. A method of administering a pharmaceutical product in the form of an aerosol according to claim 8, wherein said resistant pump synchronizes, the administration of a pre-established air flow with the expiration phase.

10. A method of administering a pharmaceutical product in the form of an aerosol according to claim 8, wherein the air flow to the aerial tracts of a patient is conveyed by a connector and a mask or mouthpiece, and said duct for conveying a pre-established air flow being operatively connected to said connector or to said mask.

11. A method of administering a pharmaceutical product in the form of an aerosol according to claim 8 wherein a flow of air is generated by said resistant pump which synchronizes the administration of a pre-established flow of air with the expiration phase and a second resistant pump which works continuously at a constant pressure during inspiration and expiration respiration phases.

12. A method of administering a pharmaceutical product in the form of an aerosol according to claim 8 wherein the second resistant pump operates at a constant pressure of 1 cm of water.
